(19) United States
(12) Patent Application Publication
(10) Pub. No.: US 2015/0126857 A1
(43) Pub. Date: May 7, 2015

(54) OPTICAL PROBE AND MEDICAL IMAGING APPARATUS INCLUDING THE SAME

(71) Applicant: Samsung Electronics Co., Ltd., Suwon-si (KR)

(72) Inventors: Min-seog CHOI, Seoul (KR); Woon-bae KIM, Seoul (KR); Seung-wan LEE, Suwon-si (KR); Eun-sung LEE, Hwaseong-si (KR); Jong-hyeon CHANG, Suwon-si (KR); Hyun CHOI, Yongin-si (KR)

(73) Assignee: Samsung Electronics Co., Ltd., Suwon-si (KR)

(21) Appl. No.: 14/479,469

(22) Filed: Sep. 8, 2014

(30) Foreign Application Priority Data
Nov. 7, 2013 (KR) 10-2013-0134988

(51) Int. Cl. A61B 1/00; A61B 5/00

(52) U.S. Cl. CPC A61B 1/00174; A61B 1/00172; A61B 5/0066; A61B 5/0095; A61B 1/00165
USPC 600/425; 600/109

(57) ABSTRACT

Disclosed are an optical probe and a medical imaging apparatus which includes the optical probe. The optical probe includes an optical scanner, which includes first and second fluids which have different refractive indexes and are not mixed with each other, and a probe body that is insertable into a coelom, and in which the optical scanner is provided in the probe body. Light which is emitted from the optical scanner is irradiated onto an object via a light output device. An output angle of the light emitted from the optical scanner varies based on a corresponding change in an interface between the first and second fluids.

### CROSS-REFERENCE TO RELATED APPLICATIONS

This application claims priority from Korean Patent Application No. 10-2013-0134988, filed on Nov. 7, 2013 in the Korean Intellectual Property Office, the disclosure of which is incorporated herein by reference in its entirety.

### BACKGROUND

1. Field

One or more exemplary embodiments relate to an optical probe and a medical imaging apparatus including the same.

2. Description of the Related Art

In the medical imaging field, the demand for information which relates to a tissue (for example, a human body or a skin) surface and technology photographing a lower tomography is increasing. In particular, most cancers occur under an epithelial cell and metastasize to inside a hypodermal cell. Therefore, when it is possible to detect cancer in its early stages, damage caused by the cancer is considerably reduced. A conventional imaging technology which uses a magnetic resonance imaging (MRI) apparatus, a computed tomography (CT) apparatus, ultrasound waves, or the like photographs an internal tomography under the skin, but because image resolution is relatively low, it may be impossible to early detect small-size cancer. Conversely, in recently proposed technologies such as optical coherence tomography (OCT) technology, optical coherence microscopy (OCM) technology, and photoacoustic tomography (PAT) technology which use light unlike the existing method, although a skin penetration depth may be as low as 1 mm to 2 mm (in the case of the OCT technology) or 50 mm to 50 mm (in the case of the PAT technology), image resolutions thereof are about ten to twenty times higher than that of ultrasound waves, and thus, are expected to be highly useful in diagnosing incipient cancer.

As described above, a medical imaging method uses a small probe that receives light from a light source and transfers the light to the inside of a human body, for inserting an endoscope, celioscope, a surgical robot, and the like inside the human body. An optical probe includes an optical lens group, which focuses light on a certain distance, and an optical scanning element that irradiates light onto a certain region.

Examples of a scanning method include a method that changes a tilt angle of a mirror in order to control a light path and a method that directly modifies an optical fiber in order to control a light path. A scanning method of a mirror changes a propagating direction of light one or more times, but is limited in reducing a diameter of a probe. Conversely, a scanning method of an optical fiber minimizes a diameter of a probe, but due to an actuator that drives the optical fiber, the length of the fiber is reduced.

### SUMMARY

One or more exemplary embodiments include an optical probe and a medical imaging apparatus including the same, which adjust an interface between fluids which have different respective refractive indexes in order to control a light path.

Additional aspects will be set forth in part in the description which follows and, in part, will be apparent from the description, or may be learned by practice of the presented exemplary embodiments.

According to one or more exemplary embodiments, an optical probe includes: an optical scanner that includes a first fluid which has a first refractive index and a second fluid which has a second refractive index which is different from the first refractive index, wherein the first fluid and the second fluid not mixed with each other; and a probe body that is insertable into a coelom, and in which the optical scanner is provided, wherein light which is emitted from the optical scanner is irradiated onto an object via a light outputter, and wherein an output angle of the light which is emitted from the optical scanner varies based on a corresponding change in an interface between the first fluid and the second fluid.

The interface between the first fluid and the second fluid may be a plane.

One of the first and second fluids may be polar, and the other may be nonpolar.

The optical scanner may be configured to one-dimensionally scan the light.

At least one of the first and second fluids may be transmissive.

The optical scanner may further include a first electrode and a second electrode that are disposed to be separated from each other with the first and second fluids therebetween, and the interface between the first fluid and the second fluid may vary based on a difference between voltages which are respectively applied to the first electrode and the second electrode.

A sum of a first contact angle between a polar fluid from among the first and second fluids and the first electrode and a second contact angle between the polar fluid and the second electrode may be substantially equal to 180 degrees.

A hydrophobic insulating layer may be formed on a respective surface of each of the first electrode and the second electrode such that the hydrophobic insulating layer is in contact with each of the first fluid and the second fluid.

At least one from among the first electrode and the second electrode may be a hydrophobic electrode.

Each of the first electrode and the second electrode may be disposed in parallel with a length direction of the probe body.

The optical scanner may further include a third electrode and a fourth electrode that are disposed to be separated from each other with the first and second fluids therebetween, and the interface between the first fluid and the second fluid may vary based on a difference between voltages which are respectively applied to the third electrode and the fourth electrode.

The optical scanner may be configured to two-dimensionally scan the light.

The optical probe may further include an optical fiber that is configured to transfer the light to the optical scanner.

The optical probe may further include a collimator that is disposed between the optical fiber and the optical scanner, and which is configured to cause the light which is emitted from the optical fiber to be substantially vertically incident onto the optical scanner.

The optical probe may further include a light focuser that is disposed between the optical scanner and the light outputter, and which is configured to focus the light which is emitted from the optical scanner onto the object.

The light focuser may include a graded index (GRIN) lens.

According to one or more exemplary embodiments, a medical imaging apparatus includes: a light source that is configured to emit light; and the optical probe that is configured to irradiate the emitted light onto an object.

The optical probe may be further configured to illuminate the object, and the medical imaging apparatus may include an endoscope.

The medical imaging apparatus may further include an optical splitter that is configured to split the light which is emitted from the light source into measurement light and reference light, to transfer the measurement light to the optical probe, and to receive response light, in response to the transfer of the measurement light, from the optical probe, wherein the medical imaging apparatus may be configured to use an optical coherence tomography (OCT) technology.

The medical imaging apparatus may further include an ultrasound transducer that is configured to convert an ultrasound wave which is emitted from the object into an electrical signal, wherein the medical imaging apparatus may be configured to use a photoacoustic tomography (PAT) technology.

### BRIEF DESCRIPTION OF THE DRAWINGS

These and/or other aspects will become apparent and more readily appreciated from the following description of exemplary embodiments, taken in conjunction with the accompanying drawings in which:

FIG. 1 is a diagram which illustrates a schematic structure of an optical probe, according to an exemplary embodiment;

FIG. 2A is a diagram which specifically illustrates an optical scanning unit of FIG. 1;

FIG. 2B is a graph which shows a relationship between a voltage, which is applied to the optical scanning unit of FIG. 1, and an output angle;

FIGS. 3A, 3B, and 3C are reference diagrams which respectively illustrate an optical scanning method which is executable by an optical scanning unit;

FIGS. 4A, 4B, and 4C are diagrams which exemplarily illustrate respective two-dimensional (2D) scanning types;

FIGS. 5A and 5B are diagrams which illustrate respective optical probes, according to exemplary embodiments;

FIG. 6 is a block diagram of a medical imaging apparatus, according to an exemplary embodiment;

FIG. 7 is a block diagram of a medical imaging apparatus, according to another exemplary embodiment; and

FIG. 8 is a block diagram of a medical imaging apparatus, according to another exemplary embodiment.

### DETAILED DESCRIPTION

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the figures, to explain aspects of the present description. Expressions such as “at least one of,” when preceding a list of elements, modify the entire list of elements and do not modify the individual elements of the list. In the drawings, the size of each element may be exaggerated for clarity and convenience of description.

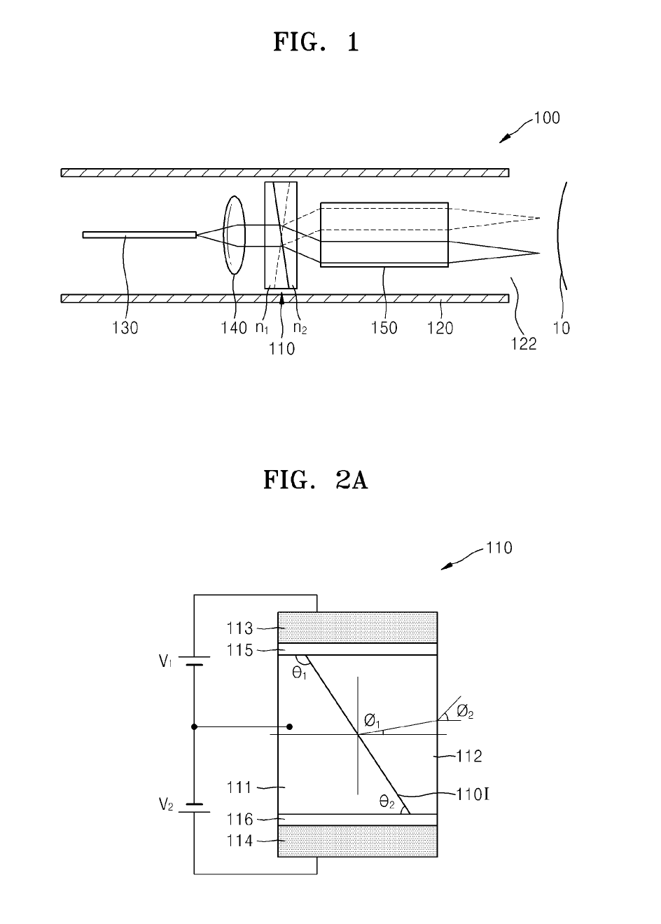

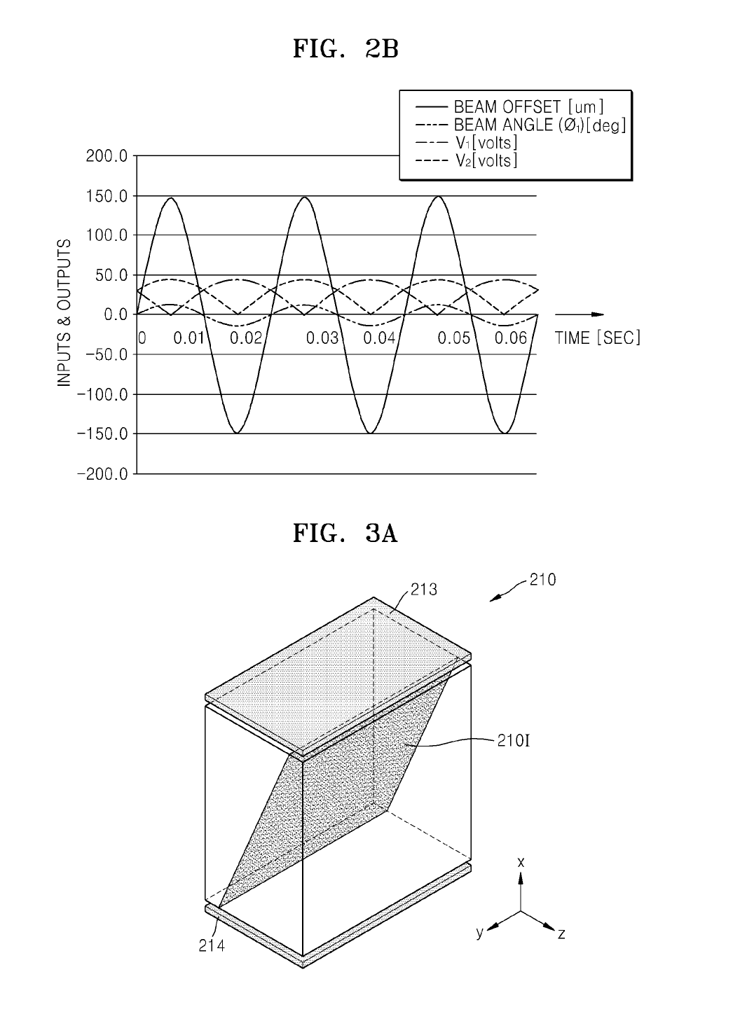

FIG. 1 is a diagram which illustrates a schematic structure of an optical probe 100, according to an exemplary embodiment. FIG. 2A is a diagram which illustrates an optical scanning unit 110 of FIG. 1. FIG. 2B is a graph which shows a relationship between a voltage, which is applied to the optical scanning unit 110 of FIG. 1, and an output angle φ₂.

As illustrated in FIGS. 1 and 2A, the optical probe 100 includes the optical scanning unit (also referred to herein as an “optical scanner”) 110, including first and second fluids 111 and 112 that have different refractive indexes and are not mixed with each other, and a probe body 120 in which the optical scanning unit 110 is provided, and light which is emitted from the optical scanning unit 110 is irradiated onto an object 10 via a light output unit (also referred to herein as a “light outputter” and/or as a “light output device”) 122. An output angle φ₂ of the light which is emitted from the optical scanning unit 110 may vary based on a corresponding change in an interface 1101 between the first and second fluids 111 and 112. The optical probe 100 may further include an optical fiber 130 that transfers light to the optical scanning unit 110.

At least one portion of the probe body 120 may be inserted into a coelom. An empty space is formed in the probe body 120, and the optical fiber 130 and the optical scanning unit 110 may be disposed in the empty space. The light output unit 122, which is opened, may be disposed in at least one region of a front end or a side end of the probe body 120. The light is irradiated onto the object 10 via the light output unit 122, or a signal (such as, for example, light, an ultrasound wave, or the like) which is reflected from the object 10 is transferred to inside the optical probe 100.

The optical fiber 130 transfers light, which is emitted from a light source (not shown), to the optical scanning unit 110. The optical fiber 130 may be disposed in parallel with a length direction (hereinafter referred to as a z-axis direction) of the optical probe 100. The light which is transferred from the optical fiber 130 may be a laser beam.

As illustrated in FIG. 2A, the first and second fluids 111 and 112 which have different refractive indexes may be disposed in the optical scanning unit 110. At least one of the first and second fluids 111 and 112 moves in an electrowetting method, and a tilt angle φ₁ of the interface 1101 between the first and second fluids 111 and 112 is changed as a result of the fluid movement. The light which is incident onto the optical scanning unit 110 is refracted at a refractive angle which varies based on the tilt angle φ₁ of the interface 1101 between the first and second fluids 111 and 112. The refracted light is refracted once more by an interface 1101 between the first fluid 111 and the outside, and is output from the optical scanning unit 110. Therefore, the output angle φ₂ of the light which is output from the optical scanning unit 110 depends on the tilt angle φ₁ of the interface 1101 between the first and second fluids 111 and 112. As the tilt angle φ₁ between the first and second fluids 111 and 112 increases, a change in width of the output angle φ₂ may increase.

The interface 1101 between the first and second fluids 111 and 112 may be a plane. Therefore, the light which is incident onto the optical scanning unit 110 may be refracted at the same angle, and may be output at the same output angle φ₂.

The first and second fluids 111 and 112 may not be mixed with each other. For example, the first fluid 111 may be formed of a polar liquid, and the second fluid 112 may be formed of a gas or a nonpolar liquid. Further, any one or both of the first and second fluids 111 and 112 may be transmissive.

The optical scanning unit 110 may further include first and second electrodes 113 and 114 that are disposed to be separated from each other with the first and second fluids therebetween. Thus, the interface 1101 between the first and second fluids 111 and 112 may vary based on a voltage difference between the first and second electrodes 113 and 114. The first and second electrodes 113 and 114 may be disposed in parallel with a length direction of the probe body 120. The first and second electrodes 113 and 114 may be transmissive, but are not limited thereto.

A hydrophobic insulating layer 115 may be formed on a surface of the first electrode 113 so as to be in contact with the first and second fluids 111 and 112, and a hydrophobic insulating layer 116 may be formed on a surface of the second electrode 114 so as to be in contact with the first and second fluids 111 and 112. However, the present exemplary embodiment is not limited thereto, and each of the first and second electrodes 113 and 114 may be a hydrophobic electrode. Therefore, when a voltage is applied to the first and second electrodes 113 and 114, a polar fluid of the first and second fluids 111 and 112 may move so that an area which is in contact with the first and second electrodes 113 and 114 and an area which is in contact with a nonpolar fluid are minimized by a surface tension. For example, when the first fluid 111 is the polar fluid, the sum of a first contact angle θ₁ between the first fluid 111 and the first electrode 113 and a second contact angle θ₂ between the first fluid 111 and the second electrode 114 may be approximately or substantially equal to 180 degrees.

A voltage Vᵢ (where i is 1 or 2, V₁ is a first voltage applied to the first electrode 113, and V₂ is a second voltage applied to the second electrode 114), which is applied to the first and second electrodes 113 and 114 and so that the sum of the first contact angle θ₁ and the second contact angle θ₂ is approximately or substantially equal to 180 degrees, may be expressed as the following Equation (1):

$${V}_{i} = \sqrt{\frac{2 γ}{c} ( cos {θ}_{i} - cos {θ}_{0} )} \\ where , \\ {θ}_{1} = 90 + 40 sin ( 2 π ft ) \& {θ}_{2} = π - {θ}_{1} \qquad ( 1 )$$

where γ [N/m] denotes a surface tension of the polar fluid of the first and second fluids 111 and 112, c denotes a capacitance (i.e., an average capacitance of the first and second fluids 111 and 112) of a fluid layer between the first and second electrodes 113 and 114, f [Hz] denotes a driving frequency of a voltage which is applied to the first and second electrodes 113 and 114, θᵢ [deg] denotes a contact angle between an electrode (the first electrode 113 or the second electrode 114) and the polar fluid of the first and second fluids 111 and 112, and θ₀ [deg] denotes a contact angle between the polar fluid and the electrode (the first electrode 113 or the second electrode 114) when the voltage is not applied to the first and second electrodes 113 and 114.

As illustrated in FIG. 2B, while the sum of the first contact angle θ₁ and the second contact angle θ₂ is being maintained at about 180 degrees, the voltage applied to the first and second electrodes 113 and 114 may be varied. Therefore, the tilt angle φ₁ between the first and second fluids 111 and 112 varies based on the voltage variations of the first and second electrode 113 and 114, and the output angle φ₂ of the light which is output from the optical scanning unit 110 varies based on the variation in the tilt angle φ₁ between the first and second fluids 111 and 112. The optical scanning unit 110 may be configured to one-dimensionally scan the light, or may be configured to two-dimensionally scan the light. A scanning method which is executable by the optical scanning unit 110 will be described below. Although not shown, the optical scanning unit 110 may further include the first and second fluids 111 and 112 and a membrane that accommodates the first and second fluids 111 and 112. A substrate, through which the light passes, of a plurality of substrates which configure the membrane, may be transmissive.

A collimator 140, which redirects the light which is emitted from the optical fiber 130 to horizontal light, may be further disposed between the optical fiber 130 and the optical scanning unit 110. The collimator 140 may be configured with one or more lenses. The horizontal light which is obtained via the redirecting by the collimator 140 may be vertically incident onto the optical scanning unit 110.

A light focusing unit (also referred to herein as a “light focuser”) 150, which focuses the light which is emitted from the optical scanning unit 110 on the object 10, may be further disposed between the optical scanning unit 110 and the light output unit 122. The light focusing unit 150 may be configured with one or more lenses. For example, the light focusing unit 150 may include a graded index (GRIN) lens that has a refractive index distribution for collecting light. The light focusing unit 150 focuses the horizontal light, which is generated by a light distributing unit (also referred to herein as a “light distributor”), onto one point of the object 10. When it is not required to focus light onto one point of the object 10, such as, for example, when the optical probe 100 simply illuminates the object 10, the light focusing unit 150 may not be an essential element.

Although not shown, the optical probe 100 may further include a bench-shaped frame that facilitates an accurate arrangement of the elements in the optical probe 100. In addition, the optical probe 100 may further include a housing or a sheath for protecting the elements which are included in the optical probe 100.

As described above, an output angle varies based on a change in an interface between different fluids having different refractive indexes, thereby reducing a length of the optical scanning unit 110. For example, the length of the optical scanning unit 110 may be reduced to about 10 mm or less. Therefore, the above-described small optical probe 100 may be applied to a medical imaging apparatus that is usable for performing diagnoses with respect to the inside of a human body. Further, because the optical scanning unit 110 does not change an optical axis of the light which is emitted from the optical fiber 130, tilting of the optical axis is small, and sensitivity to an optical axis error is lowered.

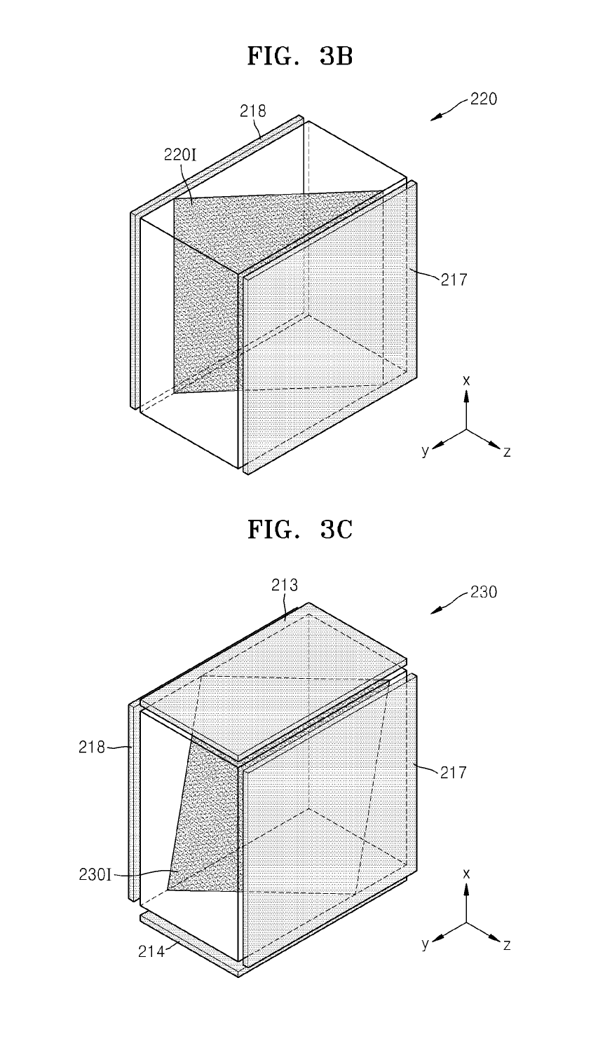

FIGS. 3A, 3B, and 3C are reference diagrams which respectively illustrate an optical scanning method which is executable by an optical scanning unit. As described above, the optical scanning unit 110 may be configured to one-dimensionally or two-dimensionally scan the light. For convenience of description, a length direction of the probe body 120 is referred to as the z-axis direction. As illustrated in FIG. 3A, a pair of electrodes (hereinafter referred to as first pair electrodes) 213 and 214 may be disposed in parallel with a z axis and a yz plane. Due to a variation in a voltage which is applied to the first pair electrodes 213 and 214, an interface 2101 between first and second fluids may swing across the z axis and with respect to an xy plane. Therefore, light which is emitted from an optical scanning unit 210 is one-dimensionally scanned in a x-axis direction.

As illustrated in FIG. 3B, a pair of electrodes (hereinafter referred to as second pair electrodes) 217 and 218 may be disposed in parallel with a z axis and an xz plane. Due to a variation in a voltage which is applied to the second pair electrodes 217 and 218, an interface 220I between first and second fluids may swing across the z axis and with respect to a yz plane. Therefore, light which is emitted from an optical scanning unit 220 is one-dimensionally scanned in a y-axis direction.

Moreover, the optical scanning unit 230 may be configured to two-dimensionally scan light. As illustrated in FIG. 3C, the first pair electrodes 213 and 214 may be disposed in parallel with the z axis and the yz plane, and the second pair electrodes 217 and 218 may be disposed in parallel with the z axis and the xz plane. Due to a variation in a voltage which is applied to the first and second pair electrodes 213, 214, 217 and 218, an interface 2301 between first and second fluids may three-dimensionally swing. Therefore, the optical scanning unit 230 may two-dimensionally scan the light.

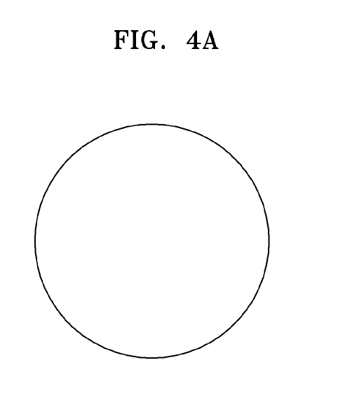

FIGS. 4A, 4B, and 4C are diagrams which exemplarily illustrate respective two-dimensional (2D) scanning types. When voltages which have the same phase and frequency are respectively applied to the first and second pair electrodes 213, 214, 217 and 218, the optical scanning unit 230 may be configured to scan light in a circular pattern type, as illustrated in FIG. 4A. When voltages which have different driving frequencies are respectively applied to the first and second pair electrodes 213, 214, 217 and 218, the optical scanning unit 230 may be configured scan light in a Lissajous pattern type, as illustrated in FIG. 4B. As another example, when voltages which have a 90-degree phase difference are respectively applied to the first and second pair electrodes 213, 214, 217 and 218, the optical scanning unit 230 may be configured to scan light in a spiral pattern type, as illustrated in FIG. 4C.

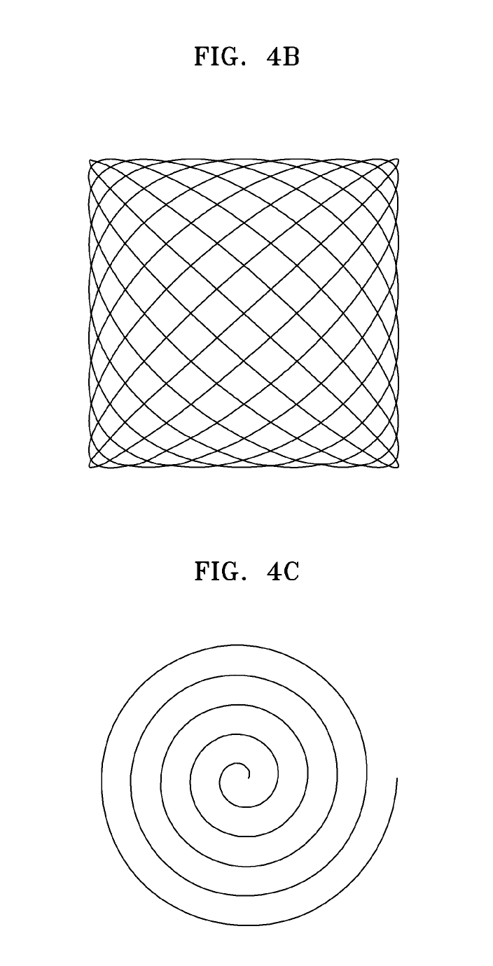

FIGS. 5A and 5B are diagrams which respectively illustrate optical probes, according to other exemplary embodiments. In comparison with the optical probe 100 of FIG. 1, an optical probe 500a of FIG. 5A may further include a light path changing unit (also referred to herein as a “light path changer” and/or a “light path changing device”) 560 that is disposed between the optical scanning unit 110 and an optical output unit 512 which are provided in a probe body 520, and an optical probe 500b of FIG. 5B may further include a light path changing unit (also referred to herein as a “light path changer” and/or a “light path changing device”) 570 that is disposed between the optical scanning unit 110 and the optical output unit 512 which are provided in the probe body 520. As illustrated in FIG. 5A, the light path changing unit 560 may be a prism. Therefore, a light path may be changed due to a total reflection of light by a surface of the prism. In addition, as illustrated in FIG. 5B, the light path changing unit 570 may be a mirror. The mirror may be a transmissive mirror or a semi-transmissive mirror.

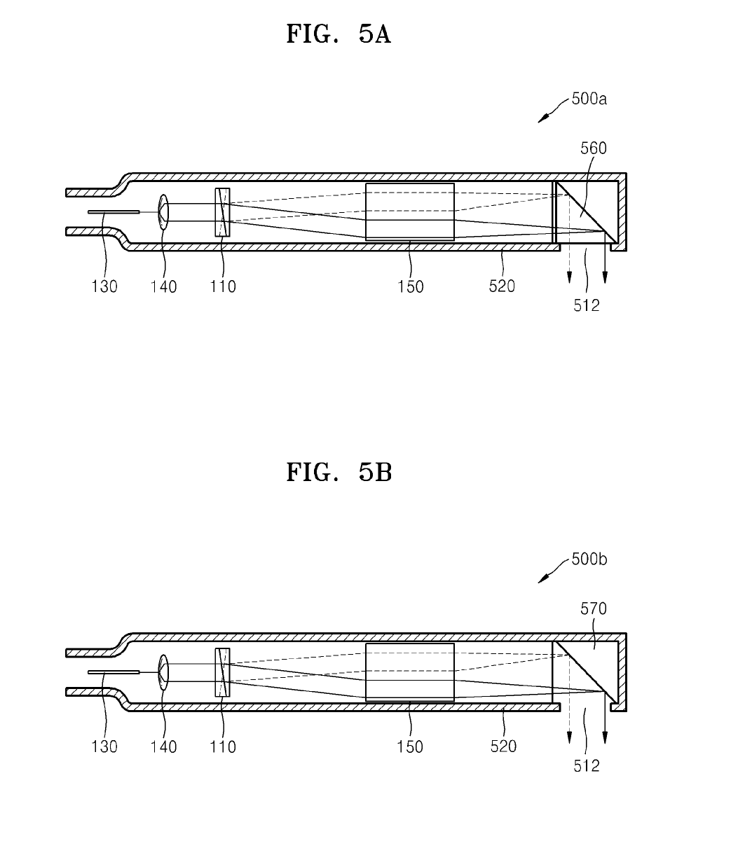

Each of the optical probes 100, 500a, and 500b may be one element of a medical imaging apparatus. For example, each of the optical probes 100, 500a, and 500b may be inserted into a coelom, and may illuminate an object. FIG. 6 is a block diagram of a medical imaging apparatus 600, according to an exemplary embodiment. The medical imaging apparatus 600 of FIG. 6 may be an endoscope. As illustrated in FIG. 6, the medical imaging apparatus 600 may include a light source 610 that is configured to emit light, an illumination unit (also referred to herein as an “illuminator”) 620 that is configured to illuminate the light onto an object 10, and a reception unit (also referred to herein as a “receiver”) 630 that is configured to receive the light which is reflected from the object 10. One of the optical probes 100, 500a, and 500b may be applied as the illumination unit 620, and the reception unit 630 may include at least one of a lens, which enlarges the light reflected from the object 10, and a photographing module that photographs the reflected light. The reception unit 620 and the illumination unit 630 may be implemented into separate probe bodies, or may be integrated into one probe body. When the reception unit 630 includes the photographing module, the medical imaging apparatus 600 may further include at least one of a signal processor, which performs signal processing on a result which is received from the photographing module to generate an image, and a display unit that displays the generated image.

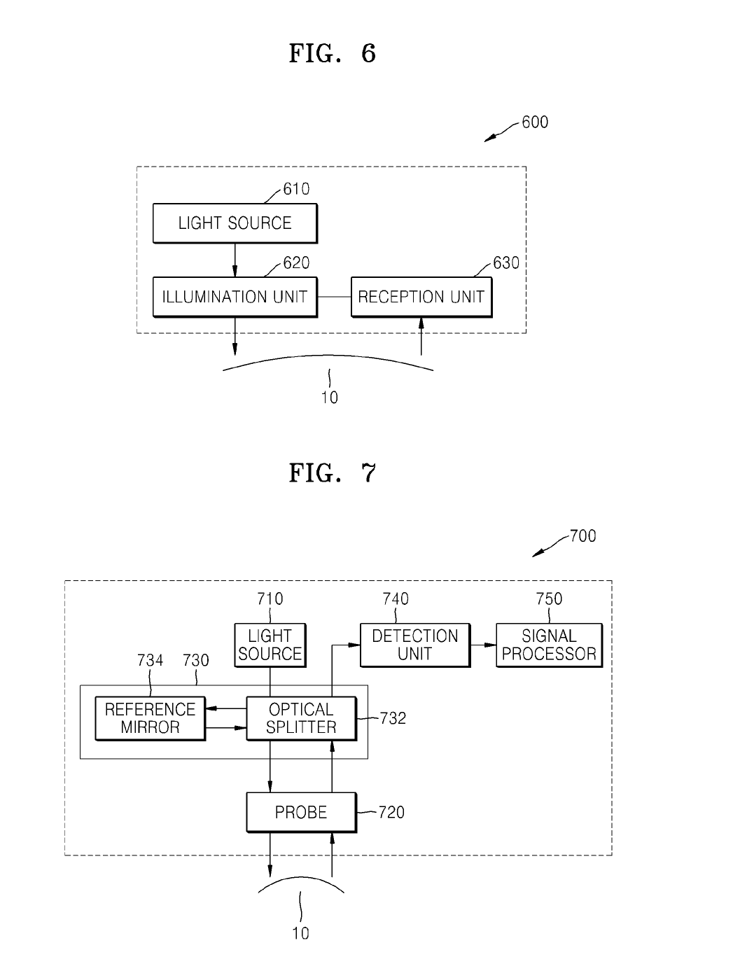

FIG. 7 is a block diagram of a medical imaging apparatus 700, according to another exemplary embodiment. The medical imaging apparatus 700 includes a light source 710 that is configured to emit light, a probe 720 that is configured to irradiate the light onto an object 10 and to receive light which is reflected from the object 10, an optical interferometer 730 that is configured to split the light which is transferred from the light source in order to apply some of the light to the probe 720 and/or to cause an interference between the light which is received from the probe 720 and reference light, a detection unit (also referred to herein as a “detector”) 740 that is configured to detect an interference signal which is applied to the optical interferometer 730, and a signal processor 750 that is configured to process the signal which is detected by the detection unit 740 in order to generate an image. In particular, the optical interferometer 730 may include an optical splitter 732 and a reference mirror 734. The medical imaging apparatus 700 of FIG. 7 may be a medical imaging apparatus to which OCT technology is applied.

An operation of the medical imaging apparatus 700 of FIG. 7 is as follows. The light source 710 emits the light, and transfers the light to the optical interferometer 730. The light transferred from the light source 710 is split into measurement light and reference light by the optical splitter 732. Among the light which is obtained as a result of the split by the optical splitter 732, the measurement light is transferred to the probe 720, and the reference light is transferred to and reflected by the reference mirror 734 in order to return to the optical splitter 732.

The probe 720 may scan a certain region of the object 10, and irradiate the light. For example, the probe 720 may be one of or a combination of the optical probes 100, 500a, and 500b. The measurement light which is transferred to the probe 720 is irradiated onto the object 10 of which an internal tomography image is to be captured by the probe 720, and the response light which is obtained from the measurement light which is reflected by the object 10 is transferred to the optical splitter 732 of the optical interferometer 730 via the probe 720. The transferred response light and the reference light which is reflected by the reference mirror 734 causes an interference to the optical splitter 732, and the detection unit 740 detects the interference signal. When the interference signal detected by the detection unit 740 is transferred to the signal processor 750, the signal processor 750 acquires an image which indicates a tomography image of the object 10 by using the interference signal. It has been described above that the probe 720 of FIG. 7 may be one of the optical probes 100, 500a, and 500b. This is merely for convenience of description, and the present exemplary embodiment is not limited thereto. The probe 720 of FIG. 7 may be divided into a first probe, which irradiates the light onto the object 10, and a second probe, which receives the light from the object 10.

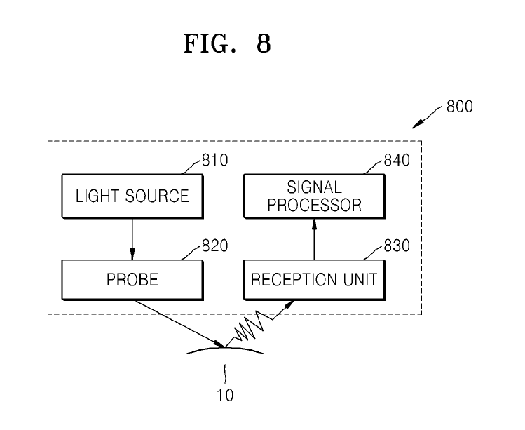

FIG. 8 is a block diagram of a medical imaging apparatus 800, according to another exemplary embodiment. Referring to FIG. 8, the medical imaging apparatus 800 includes a light source 810 that is configured to emit light, a probe 820 that is configured to irradiate the light which is emitted from the light source 810 onto an object 10, a reception unit (also referred to herein as a “receiver”) 830 that is configured to receive an ultrasound wave from the object 10, and a signal processor 840 that is configured to process a signal which is received by the reception unit 830 in order to generate an image. The medical imaging apparatus 800 of FIG. 8 may use PAT technology. PAT is a technology by which a laser pulse is irradiated into a cell tissue (an object), and a pressure wave which is generated from the cell tissue is detected in order to realize an image. When a laser beam is irradiated onto a liquid or solid material, the liquid or solid material which receives the laser beam absorbs optical energy in order to generate momentary thermal energy, which generates an acoustic wave due to a thermoelastic effect. Because an absorption rate and a thermoelastic coefficient based on a wavelength of light vary based on a material property of the object 10, ultrasound waves which have different intensities are generated from the same optical energy. By detecting the ultrasound waves, images of a distribution of blood vessels and a characteristic change of a fine tissue in a human body may be realized by a non-invasive method.

The light source 810 may include a pulse laser that induces an ultrasound wave from the object, and a pulse width may fall within a range of between approximately several picoseconds and approximately several nanoseconds.

The probe 820 may scan a certain region of the object and irradiate light onto the object, and for example, may use one of or a combination of the optical probes 100, 500a, and 500b.

When the probe 730 irradiates light onto the object 10, an ultrasound wave is generated from the object 10. Ultrasound waves having different frequency bands or intensities are generated based on a respective pulse width and a respective pulse fluence of a laser beam and a laser absorption coefficient, a laser reflection coefficient, specific heat, and a thermal expansion coefficient of the object 10. In this aspect, when a pulse laser is irradiated onto the object 10, an ultrasound wave is generated based on the type of the object 10, and by detecting the ultrasound wave, an image for determining the type of the object 10 is acquired.

The reception unit 830 may include a transducer that is configured to convert the ultrasound wave, which is emitted from the object 10, into an electrical signal. For example, the transducer may include a piezoelectric micromachined ultrasound transducer (pMUT) that converts vibration, caused by the ultrasound wave, into the electrical signal. The pMUT may be formed of piezoelectric ceramic which exhibits a piezoelectric phenomenon, a single crystalline material, and a complex piezoelectric material produced by combining the materials with a polymer. In addition, the transducer may be implemented as any one or more of a capacitive micromachined ultrasound transducer (cMUT), a magnetic micromachined ultrasound transducer (mMUT), and/or an optical ultrasound detector. The signal processor 660 may process a signal which is received by the reception unit 650 in order to generate an ultrasound image.

In the descriptions of the medical imaging apparatuses according to the exemplary embodiments, a configuration using the endoscope, the OCT, the PAT, or an ultrasound wave has been described above, but the optical probe according to the exemplary embodiments may be applied to any one or more of various medical imaging apparatuses which have a structure which uses an optical coherence microscope (OCM). In this case, a reception unit may include a suitable detection sensor based on the type of a signal generated from an object, and an appropriate image signal processing method may be used.

As described above, according to the one or more of the above exemplary embodiments, because light is scanned by using an interface between fluids having different refractive indexes, it is possible to reduce the size of the optical probe. For example, in addition to the diameter of the optical probe, the length of the optical probe is reduced. Furthermore, the optical probe may be applied to a medical imaging apparatus.

It should be understood that the exemplary embodiments described therein should be considered in a descriptive sense only and not for purposes of limitation. Descriptions of features or aspects within each exemplary embodiment should typically be considered as available for other similar features or aspects in other embodiments.

While one or more exemplary embodiments have been described with reference to the figures, it will be understood by those of ordinary skill in the art that various changes in form and details may be made therein without departing from the spirit and scope of the present inventive concept as defined by the following claims.

What is claimed is:

1. An optical probe comprising:
an optical scanner that includes a first fluid which has a first refractive index and a second fluid which has a second refractive index which is different from the first refractive index, wherein the first fluid and the second fluid are not mixed with each other; and
a probe body that is insertable into a coelom, and in which the optical scanner is provided,
wherein light which is emitted from the optical scanner is irradiated onto an object via a light outputter, and
wherein an output angle of the light which is emitted from the optical scanner varies based on a corresponding change in an interface between the first fluid and the second fluid.

2. The optical probe of claim 1, wherein the interface between the first fluid and the second fluid is a plane.

3. The optical probe of claim 1, wherein one of the first fluid and the second fluid is polar, and an other of the first fluid and the second fluid is nonpolar.

4. The optical probe of claim 1, wherein the optical scanner is configured to one-dimensionally scan the light.

5. The optical probe of claim 1, wherein at least one of the first fluid and the second fluid is transmissive.

6. The optical probe of claim 1, wherein
the optical scanner further comprises a first electrode and a second electrode that are disposed to be separated from each other with the first fluid and the second fluid therebetween, and
the interface between the first fluid and the second fluid varies based on a difference between voltages which are respectively applied to the first electrode and the second electrode.

7. The optical probe of claim 6, wherein a sum of a first contact angle between a polar fluid from among the first fluid and the second fluid and the first electrode and a second contact angle between the polar fluid and the second electrode is substantially equal to 180 degrees.

8. The optical probe of claim 6, wherein a hydrophobic insulating layer is formed on a respective surface of each of the first electrode and the second electrode such that the hydrophobic insulating layer is in contact with each of the first fluid and the second fluid.

9. The optical probe of claim 6, wherein at least one from among the first electrode and the second electrode is a hydrophobic electrode.

10. The optical probe of claim 6, wherein each of the first electrode and the second electrode is disposed in parallel with a length direction of the probe body.

11. The optical probe of claim 6, wherein
the optical scanner further comprises a third electrode and a fourth electrode that are disposed to be separated from each other with the first fluid and the second fluid therebetween, and
the interface between the first fluid and the second fluid varies based on a difference between voltages which are respectively applied to the third electrode and the fourth electrode and based on the difference between the voltages which are respectively applied to the first electrode and the second electrode.

12. The optical probe of claim 11, wherein the optical scanner is configured to two-dimensionally scan the light.

13. The optical probe of claim 1, further comprising an optical fiber configured to transfer the light to the optical scanner.

14. The optical probe of claim 13, further comprising a collimator that is disposed between the optical fiber and the optical scanner, and which is configured to cause the light which is emitted from the optical fiber to be substantially vertically incident onto the optical scanner.

15. The optical probe of claim 1, further comprising a light focuser that is disposed between the optical scanner and the light outputter, and which is configured to focus the light which is emitted from the optical scanner onto the object.

16. The optical probe of claim 15, wherein the light focuser comprises a graded index (GRIN) lens.

17. A medical imaging apparatus comprising:
a light source configured to emit light; and
the optical probe of claim 1 which is configured to irradiate the emitted light onto an object.

18. The medical imaging apparatus of claim 17, wherein,
the optical probe is further configured to illuminate the object, and
the medical imaging apparatus includes an endoscope.

19. The medical imaging apparatus of claim 17, further comprising an optical splitter configured to split the light which is emitted from the light source into measurement light and reference light, to transfer the measurement light to the optical probe, and to receive response light, in response to the transfer of the measurement light, from the optical probe,
wherein the medical imaging apparatus is configured to use an optical coherence tomography (OCT) technology.

20. The medical imaging apparatus of claim 17, further comprising an ultrasound transducer configured to convert an ultrasound wave which is emitted from the object into an electrical signal,
wherein the medical imaging apparatus is configured to use a photoacoustic tomography (PAT) technology.

21. A method for performing an optical scan using an optical scanner which is provided in a probe body, comprising:
inserting the probe body into a coelom;
emitting light from the optical scanner so as to irradiate the emitted light onto an object; and
determining an output angle of the emitted light,
wherein the optical scanner includes a first fluid which has a first refractive index and a second fluid which has a second refractive index which is different from the first refractive index, wherein the first fluid and the second fluid are not mixed with each other; and
wherein the output angle of the light which is emitted from the optical scanner varies based on a corresponding change in an interface between the first fluid and the second fluid.

22. The method of claim 21, wherein the interface between the first fluid and the second fluid is a plane.

23. The method of claim 21, wherein one of the first fluid and the second fluid is polar, and an other of the first fluid and the second fluid is nonpolar.
